(19) United States
(12) Patent Application Publication
(10) Pub. No.: US 2024/0082454 A1
(43) Pub. Date: Mar. 14, 2024

(54) ADAPTIVE AIR QUALITY CONTROL SYSTEM

(71) Applicant: Steven Paperno, Westlake Village, CA (US)

(72) Inventors: Steven Paperno, Westlake Village, CA (US); Joseph R. Adamski, Burbank, CA (US)

(21) Appl. No.: 18/244,865

(22) Filed: Sep. 11, 2023

Related U.S. Application Data
(60) Provisional application No. 63/375,370, filed on Sep. 12, 2022.

(51) Int. Cl. A61L 9/20; A61L 9/22; F24F 8/108; F24F 8/167; F24F 8/22; F24F 8/24; F24F 8/30; F24F 13/08

(52) U.S. Cl. CPC A61L 9/205; A61L 9/22; F24F 8/108; F24F 8/167; F24F 8/22; F24F 8/24; F24F 8/30; F24F 13/08; A61L 2209/12; A61L 2209/14; A61L 2209/16

(57) ABSTRACT

An AAQC may include a contaminant mitigation assembly comprising a plurality of contaminant mitigation modules, each mitigation module of the plurality of mitigation modules configured to reduce an amount of at least one class of contaminant in air flowing through the air quality control system. The AAQC system may include a UV-C lamp configured to emit a light within a treatment air path. The AAQC may further comprise a hydroxyl generator, ion generator, or a filter.

### CROSS-REFERENCE TO RELATED APPLICATIONS

This application claims priority to U.S. Provisional Patent Application No. 63/375,370, filed Sep. 12, 2022, the entire contents of each of which are hereby incorporated by reference in their entirety and for all purposes.

### FIELD

### Background

The field relates to an air quality control system.

### Description of the Related Art

Indoor spaces (and some outdoor spaces) may have poor breathable air quality due to, e.g., environmental issues (e.g., wildfires), industrial pollution, biological contamination, etc. Poor breathable air quality may be unhealthy for individuals living and/or working in locations with poor air quality. Accordingly, there remains a continuing need for improving breathable air quality.

### SUMMARY

In some aspects, the techniques described herein relate to an air quality control system including: a contaminant mitigation assembly including a plurality of contaminant mitigation modules, each mitigation module of the plurality of mitigation modules configured to reduce an amount of at least one class of contaminant in air flowing through the air quality control system; a sensor assembly configured to detect the at least one class of contaminant from among a plurality of classes of contaminants; and a control computing device in electrical communication with the sensor assembly and configured to independently control each of the plurality of mitigation modules, the control computing device configured to receive a contaminant indicator signal from the sensor assembly representative of the at least one class of contaminant, and, based at least in part on the contaminant indicator signal, transmit an activation signal to the contaminant mitigation assembly to activate at least one mitigation module to reduce an amount of the at least one class of contaminant in the air.

In some aspects, the techniques described herein relate to a system, further including a blower configured to drive air flow through the system.

In some aspects, the techniques described herein relate to a system, wherein the sensor assembly includes a plurality of sensors, each sensor of the plurality of sensors configured to detect at least one of a biological contaminant, an inorganic gas contaminant, a volatile organic compound, and particulate matter.

In some aspects, the techniques described herein relate to a system, wherein the plurality of mitigation modules includes an atmospheric water scavenger configured to scavenge water from the air flowing through the system.

In some aspects, the techniques described herein relate to a system, wherein the plurality of mitigation modules includes an ultraviolet-C (UV-C) reaction chamber configured to reduce an amount of at least one of a biological contaminant, an inorganic gas contaminant, and a volatile organic compound.

In some aspects, the techniques described herein relate to a system, wherein the plurality of mitigation modules includes an ion generator configured to reduce an amount of particulate matter from the air.

In some aspects, the techniques described herein relate to a system, wherein the plurality of mitigation modules includes an electrostatic precipitator (EP) configured to reduce an amount of at least one of a volatile organic compound or particulate matter from the air.

In some aspects, the techniques described herein relate to a system, wherein the plurality of mitigation modules includes a filter configured to reduce an amount of particulate matter from the air.

In some aspects, the techniques described herein relate to a system, further including a wireless communication module configured to transmit a status signal to a user, the status signal indicative of an air quality of an environment in which the system is installed.

In some aspects, the techniques described herein relate to a system, wherein the control computing device is configured to selectively activate and deactivate each contaminant mitigation module based at least in part on signal(s) received from the sensor assembly.

In some aspects, the techniques described herein relate to a system, wherein the control computing device is configured to determine if an activated mitigation module is not functioning and, in response, to transmit a second activation signal to the contaminant mitigation assembly to activate a second mitigation module.

In some aspects, the techniques described herein relate to an adaptive air quality control system including: a manifold including an air guide, the air guide directing air along an air pathway during operation of the adaptive air quality control system; a hydroxyl generator positioned along the air pathway and configured to scavenge water from the air; and an ultraviolet (UV) lamp positioned along the air pathway, the UV lamp configured to irradiate the air along the air pathway during operation of the adaptive air quality control system.

In some aspects, the techniques described herein relate to an adaptive air quality control system, further including a blower configured to drive air flow through the system.

In some aspects, the techniques described herein relate to an adaptive air quality control system, further including a filter, wherein with the filter is upstream of the hydroxyl generator.

In some aspects, the techniques described herein relate to an adaptive air quality control system, wherein the hydroxyl generator is upstream of the UV lamp.

In some aspects, the techniques described herein relate to an adaptive air quality control system, further including an ION generator, wherein the ION generator is downstream of the UV lamp.

In some aspects, the techniques described herein relate to an adaptive air quality control system, wherein the air guide includes one or more curved or angled portions defining a serpentine air path.

In some aspects, the techniques described herein relate to an adaptive air quality control system, wherein the air guide includes one or more curved or angled portions defining a helical air path.

In some aspects, the techniques described herein relate to an adaptive air quality control system, wherein the helical air path forms a spiral and the UV lamp is located within the spiral.

In some aspects, the techniques described herein relate to an adaptive air quality control system, wherein air guide includes a coating including polytetrafluoroethylene (PTFE) or titanium dioxide (TiO2).

In some aspects, the techniques described herein relate to an adaptive air quality control system, wherein the hydroxyl generator includes a thermoelectric cooler, a power supply, and a heat exchanger.

In some aspects, the techniques described herein relate to an adaptive air quality control system, wherein the hydroxyl generator further includes a hot heat exchanger attached to a hot side of the thermoelectric cooler and a cold heat exchanger attached to a cold side of the thermoelectric cooler.

In some aspects, the techniques described herein relate to an adaptive air quality control system, wherein the hydroxyl generator is configured to be chilled below a dew point to accumulate moisture.

In some aspects, the techniques described herein relate to a hydroxyl generator including: a thermoelectric cooler; a hot heat exchanger attached to a hot side of the thermoelectric cooler; and a cold heat exchanger attached to a cold side of the thermoelectric cooler.

In some aspects, the techniques described herein relate to a hydroxyl generator, wherein the thermoelectric cooler is a Peltier cooler.

In some aspects, the techniques described herein relate to a hydroxyl generator, wherein the hot heat exchanger or the cold heat exchanger includes a plurality of fins.

In some aspects, the techniques described herein relate to a system including the hydroxyl generator and further including a power supply configured to supply power to the thermoelectric cooler.

In some aspects, the techniques described herein relate to a system including the hydroxyl generator and further including a cooling blower.

In some aspects, the techniques described herein relate to a system claim 28, wherein the cooling blower is configured to blow air on the hot heat exchanger.

In some aspects, the techniques described herein relate to an adaptive air quality control system including: at least one ultraviolet (UV) lamp; an internal air guide including: a first passage; a second passage parallel to the first passage; and a bend passage connecting the first passage to the second passage and configured to redirect air flowing through the first passage into the second passage; wherein the UV lamp is within or exposed to the internal air guide to emit light within one or both of the first passage and second passage.

In some aspects, the techniques described herein relate to an adaptive air quality control system, wherein the UV lamp is located adjacent to the bend passage and configure to emit a light within the first passage or the second passage.

In some aspects, the techniques described herein relate to an adaptive air quality control system, wherein the internal air guide further includes a first wall, a second wall, and a third wall; wherein the first passage is defined by the first wall and the second wall; and wherein the second passage is defined by the second wall and the third wall.

In some aspects, the techniques described herein relate to an adaptive air quality control system, wherein the first wall or the second wall includes a coating.

In some aspects, the techniques described herein relate to an adaptive air quality control system, wherein the coating includes polytetrafluoroethylene (PTFE) or titanium dioxide (TiO2).

In some aspects, the techniques described herein relate to an adaptive air quality control system, further including an ion generator located downstream of the UV lamp.

In some aspects, the techniques described herein relate to an adaptive air quality control system, further including a hydroxyl generator located upstream of the UV lamp.

In some aspects, the techniques described herein relate to an adaptive air quality control system including: a manifold; at least one ultraviolet (UV) lamp located within the manifold; a helical internal air guide located within the at least manifold; wherein the helical internal air guide at least partially defines a spiral air pathway around the at least one UV lamp.

In some aspects, the techniques described herein relate to an adaptive air quality control system, wherein the helical internal air guide includes a coating.

In some aspects, the techniques described herein relate to an adaptive air quality control system, wherein the coating includes polytetrafluoroethylene (PTFE) and titanium dioxide (TiO2).

In some aspects, the techniques described herein relate to an adaptive air quality control system, wherein the at least one UV lamp is parallel to an axis of the helical internal air guide.

In some aspects, the techniques described herein relate to an adaptive air quality control system, wherein the at least one UV lamp includes four UV lamps.

### BRIEF DESCRIPTION OF THE DRAWINGS

FIG. 1 is a schematic system diagram of an adaptive air quality control (AAQC) system, according to various embodiments.

FIG. 2 is a schematic system diagram illustrating the connectivity of the AAQC system of FIG. 1.

FIG. 3 is a perspective view of an embodiment of the AAQC unit.

FIG. 4 is a perspective view of an embodiment of the AAQC unit.

FIG. 5 is a perspective view of an embodiment of the AAQC unit.

FIG. 6 is a perspective view of an embodiment of the AAQC system of FIG. 1.

FIG. 7 is perspective view of an embodiment of the AAQC unit.

FIG. 8 is a view of components of the AAQC unit shown in FIG. 7.

FIG. 9 is a view of components of the AAQC unit shown in FIG. 7 with the internal air guide shown in a transparent state.

FIG. 10 is a view of an embodiment of components of the AAQC unit.

FIG. 11 is a perspective view of the front of an alternative embodiment of the AAQC unit.

FIG. 12 is an exploded view of the AAQC unit shown in FIG. 11.

FIG. 13 is a perspective view of the back of the AAQC unit shown in FIG. 11.

FIG. 14A is a cutaway side view of an embodiment of the AAQC unit.

FIG. 14B is a cutaway side view of the AAQC unit shown in FIG. 14A with arrows indicating airflow through the AAQC unit.

FIG. 15A is a schematic sectional view of an embodiment of the AAQC unit.

FIG. 15B is a schematic sectional view of the AAQC unit shown in FIG. 15A with arrows indicating airflow and UV-C radiation path through the AAQC unit.

FIG. 16A is a view of an embodiment of the AAQC unit.

FIG. 16B is a view of the AAQC unit shown in FIG. 16A.

FIG. 16C is a view of the AAQC unit shown in FIG. 16A with arrows indicating airflow.

### DETAILED DESCRIPTION

### I. Overview

Many air filters and/or cleaners are available for commercial and residential use for filtering and/or cleaning the air. For these appliances, air is typically drawn through a filter and discharged back into the living or workspace. Some air filters/cleaners use a Minimum Efficiency Reporting Value (MERV) 12 filter. This filter removes 80% to 90% of the 1.0-to-3.0-micron particulate matter, and 90% or better of the 3.0-to-10-micron material from the breathable air reservoir. Still others pair this filter with combinations of germicidal lamps, titanium dioxide (TiO2) photocatalyst, and activated charcoal to try to eliminate contaminants found in the air in indoor areas, such as classrooms, offices, and living rooms.

Outdoor air can also affect the indoor air quality. In fact, indoor air is found to be more than 10 times more contaminated than outdoor air. Typical air cleaning products fall woefully short in addressing current and new contaminants, and do not notify the appliance owner about poor air quality issues.

With every new contaminant, current air filtering appliances become less and less effective. Various embodiments disclosed herein provide a system which can adapt its functionality to address changing air contamination in real-time, which can significantly improve air quality and the health of occupants. Various embodiments disclosed herein can beneficially monitor air quality and implement corrective action by: (1) measuring air quality; (2) making intelligent choices regarding how to handle the detected contaminants; (3) applying those choices to measurably improve air quality by reducing or removing the identified contaminant; and (4) monitoring and reporting the resultant air quality to ensure that the mitigation approach is successful.

In various embodiments, the system can adapt its performance to reduce the contaminants found in breathing air in real-time. As an adaptive device, it can reduce costs spent on personal health care and the wasteful purchase of ineffective air handling products and their maintenance.

Conventional devices are not suitable to solve these air quality problems at least because, e.g., such devices do not have adequate sensing and control systems. To identify the source(s) that are causing poor air quality, various embodiments can be configured to directly measure a plurality of (e.g., 12-24) specific air contaminants. For example, various embodiments disclosed herein can detect and/or monitor the presence of biological contaminants, inorganic gas contaminants, volatile organic compounds (VOCs), and/or particulate matter contaminants (e.g., PM₁₀, PM_{2.5}).

Various electronic sensors can be designed to detect a particular type of contaminant to quantify the level of contamination detected. This contamination information (detected, for example, in parts-per-million, or ppm) can be used by an on-board control computing device (also referred to herein as a control system) to determine how best to handle the contamination. For example, the computing device can comprise processing electronics (e.g., one or more processors configured to execute instructions stored on one or more memory devices) configured to execute instructions that select one or a combination of control modalities to mitigate the contaminant threat and report progress toward a clean air goal. The control computing device can be configured to independently control each of a plurality of mitigation modules.

These control modalities include, but are not limited to, germicidal ultraviolet lamps (UV-C), titanium dioxide (TiO2) photocatalyst, activated carbon filtration, sodium bicarbonate/lime and variants of lime, sodium permanganate treated activated carbon, electrostatic precipitators, and positive and negative ion generators.

### II. System Components

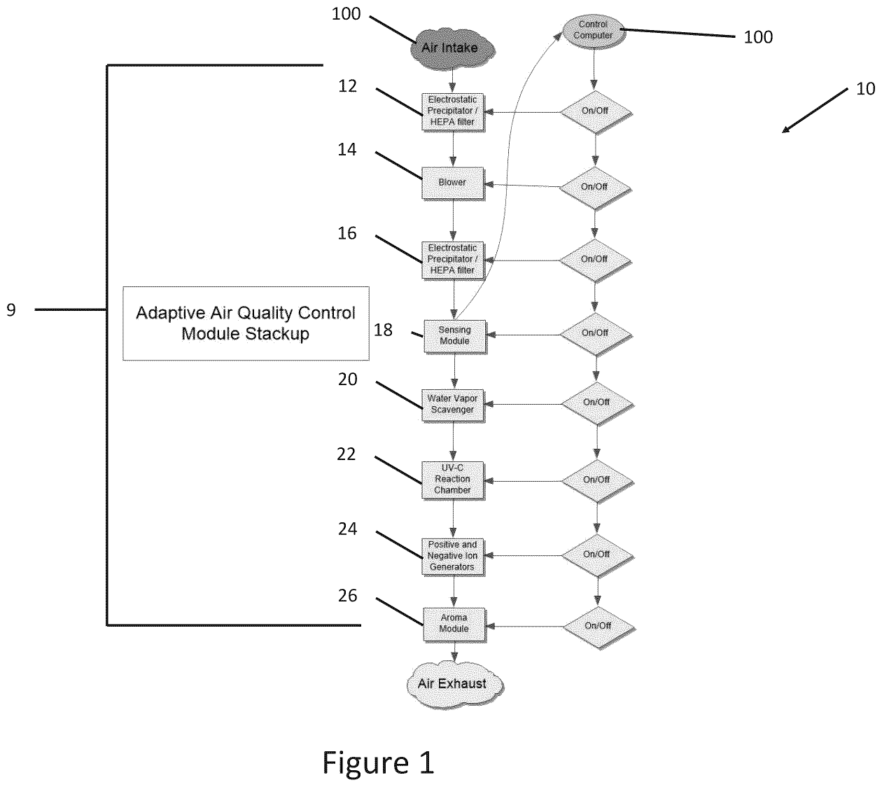

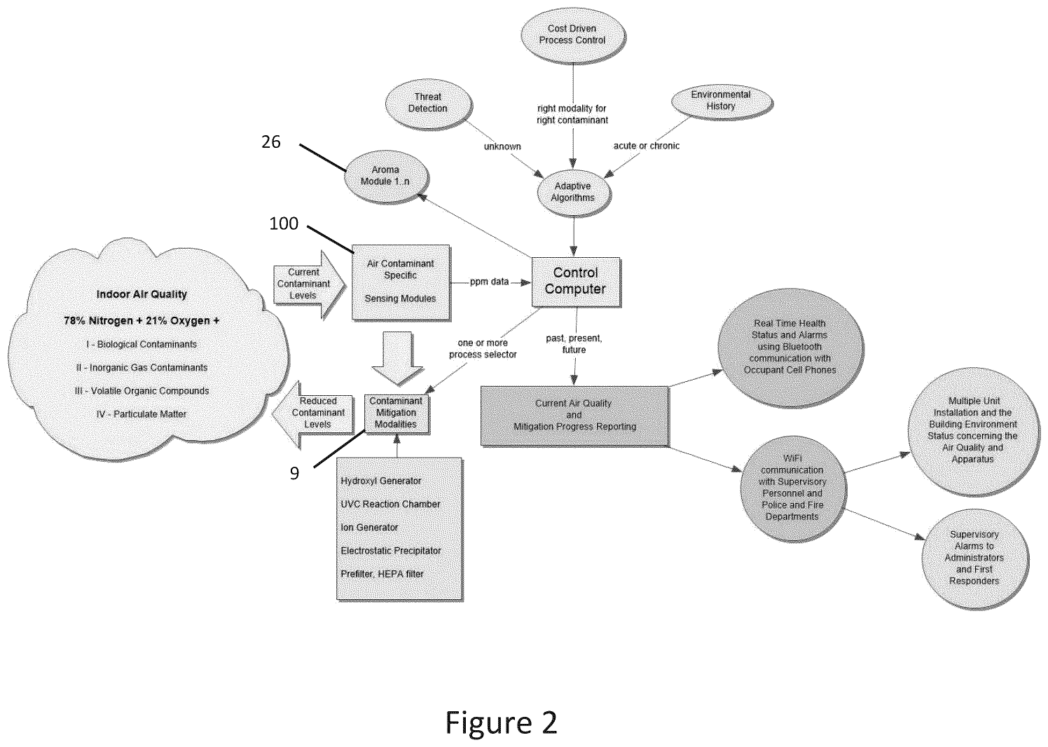

FIG. 1 is a schematic system diagram of an adaptive air quality control (AAQC) system 10, which may also be referred to as an AAQC unit, according to various embodiments. FIG. 2 is a schematic system diagram illustrating the connectivity of the AAQC system of FIG. 1. As shown in FIG. 1 and FIG. 2, the system 10 can comprise a plurality of components in electrical and/or data communication with a control computing device 100.

As shown in FIG. 1, in some embodiments, input air may be filtered using a prefilter in combination with a high efficiency particulate air (HEPA) filter, just upstream of the blower as shown in block 12 or just downstream of the blower as shown in block 16. The system may be assembled into a generally round tubing or rectangular duct configurations.

The system can comprise a blower, which comprises a primary air mover that draws air into the system as shown in block 14. The primary air mover can comprise a quiet, high CFM (cubic feet per minute) blower. Accompanying sections of the device contain specific air treatment modalities described below.

The system can comprise a contaminant mitigation assembly comprising a plurality of contaminant mitigation modules 9, each mitigation module of the plurality of mitigation modules configured to reduce an amount of at least one class of contaminant in air flowing through the air quality control system.

The contamination mitigation assembly can comprise a mitigation module 9 including an atmospheric water scavenger as shown in block 20. The atmospheric water scavenger can comprise a hydroxyl generator formed from an arrangement of germicidal UV-C lamps and a TiO₂ (titanium dioxide) photocatalyst. Water molecules can be split to create oxidizing OH molecules effective at breaking down VOCs.

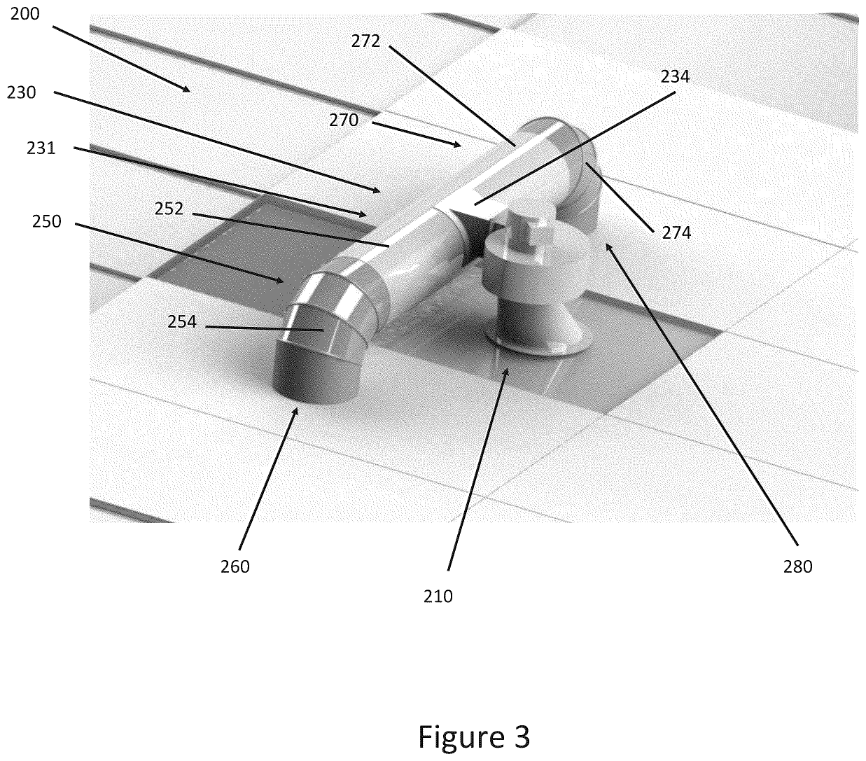

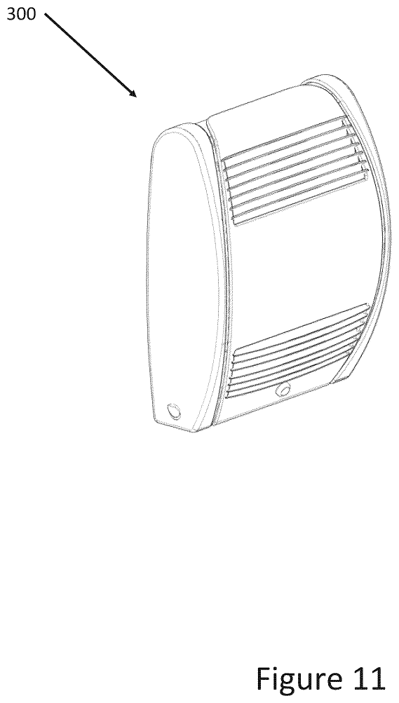

To avoid supplying water to the unit, an atmospheric water condensing or scavenging module can be provided. The atmospheric water condensing or scavenging module can comprise a thermoelectric (e.g., Peltier) cooler, a direct current (DC) power supply, and hot and cold heat exchangers, to cooperate to create a chilled surface. This chilled surface can operate below the dew point temperature (typically 40-50° F.) of the incoming air stream. Air flow around the cold heat exchanger can be tailored so that enough condensate is produced and retained for the process. The room air humidity can be scavenged into liquid water or ice forming on the fins of the cold heat exchanger. This condensate can be released back into the room under computer control to supply water vapor, and, through the photocatalytic process energized by UV-C radiation, creates hydroxyls on demand. The water condenser unit of FIGS. 1-2 may comprise any of the hydroxyl generators 212, 312 associated with AAQC unit 200 show in FIGS. 3-10 or AAQC unit 300 shown in FIGS. 11-16C.

The contamination mitigation assembly can additionally or alternatively comprise a mitigation module including an ultraviolet-C (UV-C) reaction chamber with an air vortex generating auger as shown in block 22. The ultraviolet reaction chamber of FIGS. 1-2 may comprise any of the UV-C lamps, treatment air paths, surface coatings, or related components associated with AAQC unit 200 show in FIGS. 3-10 or AAQC unit 300 shown in FIGS. 11-16C.

Air flow through the duct or tube configurations can begin as primarily axial (e.g., through the length of the tube or duct). However, it can be important to create conditions for a killing dose of radiation to render harmless dangerous biological contaminants. Since the killing dose is a mathematical product of Radiation Power Density and Dwell Time, either power density (in physical units of watts/square meter) can be increased using multiple, powerful UV-C sources, or Dwell Time (in physical units of seconds) can be increased by forcing the contaminated air to travel a longer path.

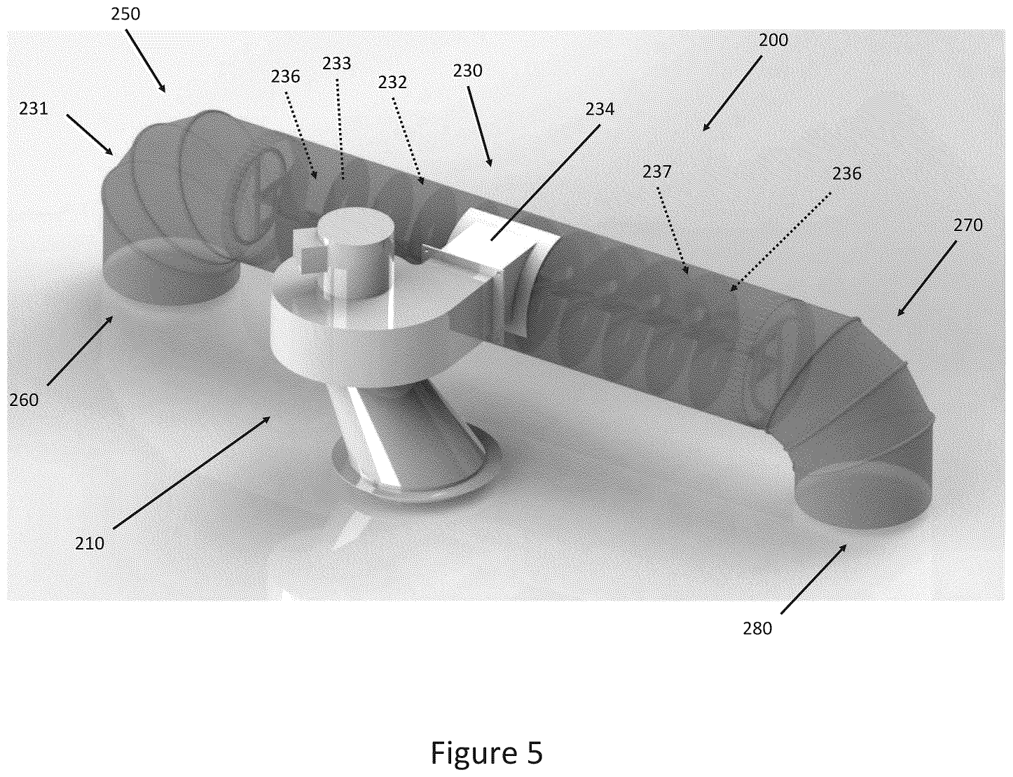

The air vortex generating auger of the disclosed embodiments as shown in FIGS. 5 and 7-9, solves several important problems. First, the air vortex generating auger (e.g., helical flow pathway) has an air flow direction transformer that works with the variable pitch of the first few auger flights, to convert axial air flow into tangential air flow. Without this air flow vector change, it may be extremely difficult to send air through the long, spiraling UV-C radiation exposure path.

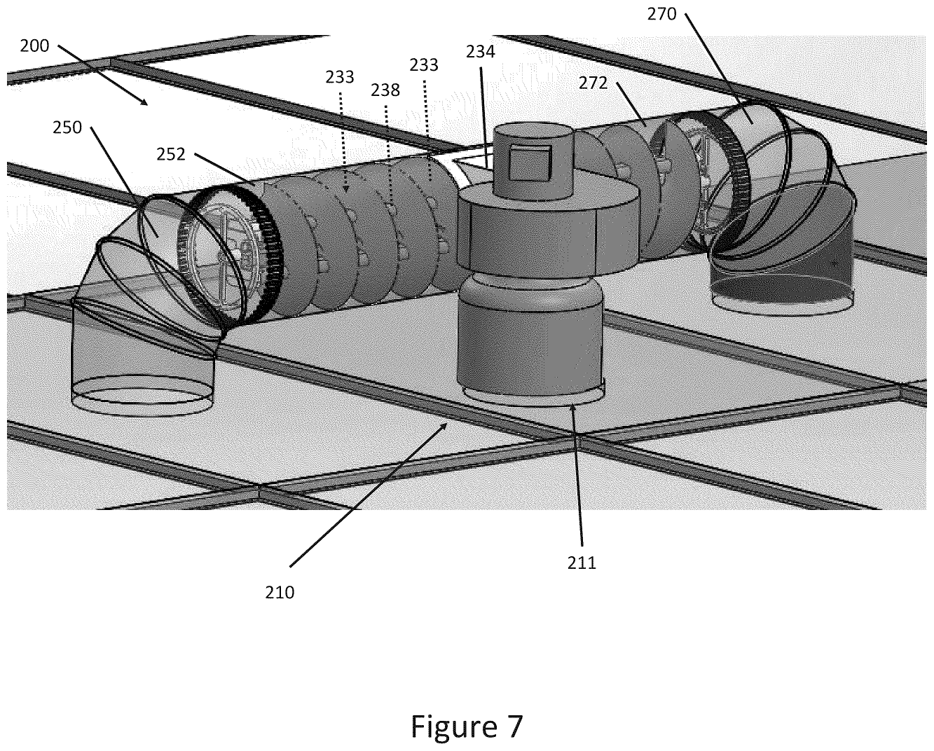

UV-C generating lamps 228 can be positioned at the axis of the auger 233, such that the tangential flow of contaminated air around these radiation sources effectively exposes biological contaminants to UV-C radiation as shown in FIGS. 7-9.

The auger may be a snap-together auger and may be used in the AAQC unit 200 as shown in FIGS. 3-10. The snap-together auger surfaces can be made of various materials. If the auger surfaces are constructed as injection molded or vacuum formed plastic parts, they can be metallized to protect the plastic from the deleterious effects of UV light and photocatalytic action of the TiO2. Alternatively, if the auger surfaces are fabricated from thin metal, the parts can be easily stamped (they are thin) and easily assembled using bend-over tabs to bind each half flight to another half flight.

The metallized coating discussed above reflects UV-C radiation back to the interior walls. These walls can be lined with high UV-C reflectivity (98%) polytetrafluoroethylene (PTFE) and titanium dioxide (TiO₂) surface coatings.

The resulting multiple reflections create a killing crossfire of radiation to inactivate biological contaminants and support photocatalytic break down of inorganic gas contaminants and volatile organic compounds.

The auger can increase the air flow path length to be up to 15 times longer that the path of the physical tube. The increase, by a factor of 10-15, in air flow path, which may also be referred to as an increase in dwell time, through the UV-C radiation source created by the auger, increases dose by a factor of 10-15. In comparison, if the system were using 2 lamps, adding an additional UV-C lamp (three total) would only increase the dose contribution by 1.5, and adding two lamps (four total) would only increase the dose contribution by 2.

For typical breathing air processing systems, the geometry of surfaces close to the lamp, the lamp specification, and the inverse square law provide a good handle on how to determine and use the lamp power for germicidal purposes. The inverse square law applies to emitters that are point sources. In these arrangements, the power measured at a distance away from the point source is inversely proportional to the square of the distance. For example, if a power level of 16 watts is measured at 2 inches from the energy source, at twice the distance away (4 inches) the power will be ¼ of the initial measurement (in this case, 4 watts). In this point source example, which is typical of light emitting diodes that emit germicidal radiation, substantial optics may be utilized to handle the significant power reduction that occurs during the inverse square energy broadcast.

Geometric optics containing highly reflective surfaces (measured at the wavelength of the incident radiation) play an important role in the design of germicidal energy applicators. In the AAQC system disclosed within, long path, closely spaced surfaces function as waveguides to guarantee that the expensive UV-C energy is best used to inactivate contaminants. The closely spaced surfaces can be thought of as two planes separated by a gap, in some cases a gap of 1 to 2 inches, into which UV-C energy is released. In this geometry, energy emitted between the planes does not follow the inverse square law, because grazing angle incidence at the planar surfaces contributes to higher reflection energy. Instead of radiating out into space, (where the illuminated area increases as the square of the distance, the brightness of the light must decrease as the inverse square), the energy is guided in the parallel sheets, providing significant increase in power (watts) available to destroy pathogens farther from the source.

An additional component of the AAQC system introduces air movement into the dose equation. As the goal is to process as much contaminated air as quickly as possible, the disinfection system receives a moving air volume, created by on-board fans or blowers. These design components have similar size and shape restrictions to deliver a CFM (cubic-feet/minute) flow rate that directly impacts the number of Air Changes per Hour delivered by the system. While not governed by the Air Changes Per Hour (ACH) heuristic, the systems disclosed herein can effectively be compared to typical air moving systems using the number of Air Changes per Hour that other products deliver.

The embodiments described herein may include a cylindrical auger shaped flow path as shown in FIGS. 5 and 7-9 which applies germicidal radiation over the complete air volume path. In that embodiment, the cylindrical path increases the dose by 10 to 20 times.

In additional embodiments shown in FIGS. 11-16C, path length increases of 2 or 3 or more times are possible using a serpentine arrangement of air flow channels and radiation sources which shine inactivation energy down between the channels. These channels can be constructed of very low-cost materials, ranging from aluminized plastic film, to injection molded panels, to thin aluminum sheet metal panels with injection molded and metalized return curves. The accompanying figures show various ways to create long path length applicators.

An advantageous side effect of the energy (E=hν) of UV radiation (e.g., radiation having a wavelength in a range of 100 nm to 400 nm, e.g., about 254 nm) radiation is that exposing moisture or water droplets to radiation at UV wavelengths creates —OH radicals. These radicals contribute to the destruction of long chain organic contaminant molecules present in breathing air, most of which are too dangerous for human consumption. The generation of the OH radicals occurs during the interaction of the UV energy with water molecules present in the ambient atmosphere. It should be understood that the wavelength need not be exactly 254 nm, for example the wavelength may be between 100 nm and 400 nm, or between 200 nm and 300 nm. The reaction rate can be accelerated using a titanium dioxide catalyst, in the case of AAQC system, applied to the waveguide walls as described herein.

The chemical reaction rate at which OH radicals break down organic contaminants is proportional to the availability of water molecules. In certain locations, the very low humidity conditions may not be conducive to plentiful OH radical formation. As such, embodiments of the AAQC systems, such as AAQC unit 200 and AAQC unit 300, include an integrated water molecule source, without needing to connect the unit to a water supply source. The integrated water molecule source can be referred to as a hydroxyl generator. The hydroxyl generator can comprise an arrangement of Peltier plates and heat exchangers that are used to condense liquid water from the atmosphere. The hydroxyl generator may be controlled by the control computer.

Since the temperature of the fins of the cold heat exchanger are below the dew point, condensate forms on the fins. The condensate can be used directly from the fins or collected in a drip pan and stored (as a water capacitor) when the unit is operating as an air sterilizer, or during temporary high humidity ambient conditions, for example, conditions that occur during kitchen pasta pot boiling, or from a bus stop load of kids headed for school that arrive at the classroom.

These temporarily humid air streams contain additional H₂O molecules that can be condensed and stored in this rehydration module. Combined with the high power ultraviolet UVC radiation, and a Light-Activated Catalytic Oxidizer (LACO) (nanosized anatase crystal titanium dioxide), airborne and scavenged water molecules are transformed into hydroxyl OH radicals, which can destroy or break apart long chain organic contaminants.

The titanium dioxide LACO, OH radicals, high power UV-C radiation and long irradiation path length break the atomic bonds of organic and inorganic contaminants, delivering a single pass kill dose, inactivating and destroying a wide range of viruses, bacteria, and other pollutants in the process. A single pass kill dose can comprise a single pass of air through a device that is able to remove or kill all or substantially all (e.g., at least 90%, at least 95%, at least 97%, at least 98%, or at least 99% of contaminants) contaminants (e.g., viruses, bacteria, and other organic and inorganic contaminants) in the air. Traditional systems rely on recirculating air through a system multiple times, killing 70% each time, until the room reaches an acceptable level. The single pass kill system is facilitated by a substantial dwell time which allows the AAQC to increase the percentage of viruses, bacteria, and other pollutants removed per air cycle. The dwell time is increased by creating a longer path for the air flowing through the system. This increase in air path length and dwell time is accomplished by components such as internal air guide 332, which comprises the baffle 333. A dwell time may be between 0.1 and 1 second in some embodiments.

In the final processing output stage of embodiments of the AAQC, negative ions and OH radicals are discharged back into the breathing space to help ensure pathogens and contaminants continue to be destroyed or rendered harmless farther away from unit.

The AAQC system is configured to destroy SARS-CoV-2 and other viruses, bacteria, fungi, mold, spores, CO carbon monoxide, CO₂ carbon dioxide, NO₂ nitrogen dioxide, SO₂ sulfur dioxide, O₃—ozone, HCHO—formaldehyde, TVOC—total volatile organic compounds, PM2.5-2.5 micron particulate matter, and PM10-10 micron particulate matter. It should be understood that the system may destroy or sanitize additional air contaminants beyond those listed.

As shown in FIG. 1, the contamination mitigation assembly can additionally or alternatively comprise a mitigation module including an ion generator as shown in block 24. To help improve air quality, the ion generator module can comprise both positive and negative ion sources that can be selectively operated. The ion generator can be activated when the particulate matter sensors inform the control computer that it detects particulate matter. The ion generator primarily serves to electrically charge contaminants present in the air flow stream, so they can be plated out onto rugs, walls, windows, ceilings, countertops, and furniture surfaces. When the contaminants are attracted to and retained on these surfaces, the contaminants are no longer present in the breathable air reservoir.

An additional benefit of the positive and negative ion generation capabilities of the AAQC system is that negative ions can be broadcast on an as needed basis or “by the timeclock” to create a “feel good” environment attributed to the ions in real-time. This capability is especially useful in school classrooms, business offices, meeting rooms, etc.

The contamination mitigation assembly of FIGS. 1 and 2 can additionally or alternatively comprise a mitigation module 9 including an electrostatic precipitator (EP). The electrostatic precipitator can comprise a multi-part (e.g., two-part) device, which creates free space electrons which collide with contaminants, leaving the contaminants with a net (non-zero) charge. Metal plates of appropriate negative or positive net charge inside the EP attract and retain these charged contaminants—effectively removing them from the air stream. These EP plates can be cleaned periodically. In some operating scenarios, the EP can be an effective alternative to the use of HEPA filters for air quality control. For example, when the HEPA filters have reached end of life and are no longer useful, the EP can take over.

In some configurations, an activated carbon honeycomb, with selectively coated TiO₂ and metallized surfaces can be used in the EP. This assembly serves multiple purposes—e.g., electrostatic precipitation, adsorption of VOCs on bare carbon surfaces of the honeycomb and photocatalytic reduction of VOCs by the TiO₂ surfaces.

The contamination mitigation assembly can additionally or alternatively comprise a mitigation module including a prefilter, such as a HEPA filter. HEPA filters can work to remove 99.97% of 0.3 micron to 10-micron particulates from air. The design and location of these filters in the configuration takes advantage of the blower air handling performance (e.g., flow rate and pressure) to create useful air changes per hour in the living spaces being treated.

In some embodiments, the contamination mitigation assembly can be configured with redundancy such that, if one mitigation module is consumed or breaks, then another mitigation module can be activated in its place. For example, if the EP is not functioning as expected the control computer 100 can activate one or more additional mitigation modules, such as the ion generator or the HEPA filter. In this example, if the control computer 100 determines that the EP is not functioning, the control computer can activate one or more of the ion generator and HEPA filter to perform the charging and removal of contaminants. Thus, instead of depositing contaminants onto plates of the EP, the contaminants may instead be deposited onto walls, ceilings, floors, etc. by the ion generator.

As shown in FIGS. 1 and 2, the system 10 can include sensor modules as shown in block 18 and a control computing device 100, which can cooperate to monitor contaminants and control the operation of the Adaptive Air Quality Control (AAQC) unit.

The control computer can comprise processing electrics configured to assemble data from the sensors, determine through analysis if the data represents a chronic or acute event, activate any number of contamination-mitigating elements discussed above, and monitor and report air quality resulting from the application of these mitigating elements.

An additional characteristic of the control computer is that, as shown in FIG. 2, the system can comprise a wireless communications module configured to enable the control computing device to communicate wirelessly, for example, via Bluetooth (for short range data transfer between devices) or WiFi (which allows devices to connect to the internet).

These two features can cooperate to wage a real time battle against potentially dangerous contaminants present in the air which we breathe.

An additional benefit of the computer-based monitor and just-in-time application of specific modalities to address specific contaminants, is that the computer can accurately totalize “on-time” of a filter or a lamp or an ion generator—and transmit that data to a maintenance supervisor who can specifically address replacement issues for those components. For example, the control computing device can transmit instructions to the control modalities to selectively turn one or more of the modalities on or off for a specified time period.

The on-board computer can also estimate and report how long it might take to handle an influx of contaminated air, such as when the living room door was opened and a breeze of smoke laden air from a distant wildfire entered the living space.

As discussed above, the sensing modules can measure amounts of and/or detect at least four contaminant categories—biological contaminants, inorganic gas contaminants, volatile organic compound contaminants and particulate matter contaminants.

Some sensors can be configured to detect biological contaminants in real time. For example, some sensors can detect the presence of viral, bacterial, fungal and mold/spore contaminants for purposes of warning/quarantining individuals or groups. The control computing device can utilize the real-time data from these sensors to control air quality. Skilled artisans will appreciate that any suitable sensors can be used to detect the contaminants. As an example, various embodiments can utilize a carbon monoxide sensor, such as a model TGS5141 sensor sold by Figaro USA, Inc. of Arlington Heights, Illinois. As another example, various embodiments can utilize an indoor air quality sensor platform, such as a ZMOD4410 Gas Sensor Module sold by Renesas Electronics Corporation of Tokyo, Japan.

The inorganic gas contaminant detection module can include a sensing module 18 including sensors to quantitatively measure at least the following inorganic gas contaminants: carbon dioxide, carbon monoxide, nitrogen oxides, ozone, sulfur dioxide, and ammonia. It should be appreciated that the inorganic gas contaminant detection module can additionally or alternatively be configured to detect other types of inorganic gases.

Additionally, a VOC contaminant detection module, as well as specific sensors to quantitatively measure these volatile organic compounds, can be configured to measure amounts of and/or detect at least benzene, ethylene glycol, formaldehyde, methyl chloride, tetrachloroethylene, toluene, xylene, 1,3 Butadiene, and acetaldehyde. It should be appreciated that the VOC contaminant detection module can additionally or alternatively be configured to detect other types of VOCs.

One or more particulate matter (PM) contaminant detection modules can comprise sensors for PM₁₀ and PM_{2.5} level contaminants used to identify sulphates, nitrates, elemental carbon, organic carbon and earth/ash for capture and destruction using GUV-C+TiO2 and electrostatic precipitation.

For each of the sensor modules, alarms can be set to warn occupants, supervisor personnel and first responders that a potentially hazardous condition exists around one of the installed AAQC units. Operating in conjunction with the wireless communication module (e.g., Bluetooth and WiFi communication components) of the control computer, this data serves to inform occupants and advise administrative personnel or software as to the precise area of contamination—e.g., a contamination in a 3^{rd} floor chemistry lab, or as a result of a deliberate arson.

As compared with some conventional systems, the Adaptive Air Quality Control system can beneficially acquire contaminant measurements in real-time during operation of the unit. These measurements, addressing multiple (e.g., four) categories of contaminants as discussed above, can be processed by the control computer, and used to activate or deactivate one or more air quality improvement modalities. For example, if biological sensors do not detect a hazardous contaminant, the GUV-C lamps can be cycled off—and air quality maintenance may continue using activated carbon or HEPA filters.

Continuing with potential operating examples, a kitchen range or cooktop or forced air heating system for the home might exhibit a problem with combustion and create high levels of hazardous carbon monoxide. The Adaptive Air Quality Control system can react to this new condition by exposing once sealed activated carbon media to the air stream, effectively reducing the ppm (parts per million) concentration of carbon monoxide (CO), as measured by the on-board CO sensor, to satisfactory levels. In this manner, the AAQC system adapts to and reports the change in air quality.

As another example, the State of California is experiencing unprecedented water shortages and wildfires resulting from extremely long periods of time with no rain. Smoke from wildfires is creating its own weather patterns as well as severely impacting air quality in affected areas across the United States. The AAQC system can beneficially measure the Particulate Matter level and as appropriate, activate either the electrostatic precipitator or direct air flow through the HEPA filter or Activated Carbon Filter. When viewed from an economic standpoint, the use of the EP may be more cost effective than using the HEPA filter; the AAQC unit computer control can take advantage of these types of algorithms to deliver the best air quality at the best affordable price if desired.

The on-board control computer can also monitor air temperature, humidity, UV-C power output of the germicidal lamps, air flow/velocity, ion generator performance, etc., to advise the maintenance supervisor of impending or actual problems with the basic hardware of the AAQC.

As shown in FIGS. 1 and 2, the system can comprise one or more aroma adjustment modules as shown in block 26. These modules support the creation, under computer control (with all its communication abilities) of a wide variety of scents. These scents can be applied to fill selected rooms/meeting areas with Pine, Coffee, Lavender, Summer Breeze, Sea Breeze, Vanilla, Orange, Grapefruit and Lemon, Mint, Fresh Cut Grass, Night blooming Jasmine, Himalayan Salt, and New England Maple or Cinnamon scents—to complement the living space and occupant activity as desired. Other scents may be suitable.

The system components can be manufactured in any suitable manner. As an example, various sensors which can be used in the AAQC unit may be manufactured by Figaro Engineering, Inc. of Arlington Height, IL. The blower can comprise any suitable type of blower such as a high pressure, low noise Dayton blower manufactured by W.W. Grainger, Inc. of Lake Forest, Illinois, or a FASCO blower, sold by BECK America, Inc. of Muscle Shoals, Alabama. Any suitable thermoelectric cooling device can be used, such as thermoelectric cooling devices available from Ferrotec Holdings Corporation of Tokyo, Japan, including the Peltier cooler and/or a full thermoelectric cooling assembly including heat sinks. The water vapor scavenger/hydroxyl module can use any suitable power supply such as a supply sold by Mean Well USA, Inc. of Fremont, California. Any suitable low-cost germicidal UV-C lamps and ballasts, ion generators, activated carbon and HEPA filters can be provided. The control computing device can comprise any suitable number and type of processing electronics, including one or more processors and memory devices that can communicate with and control the operation of the sensor modules and the operation of the control modalities.

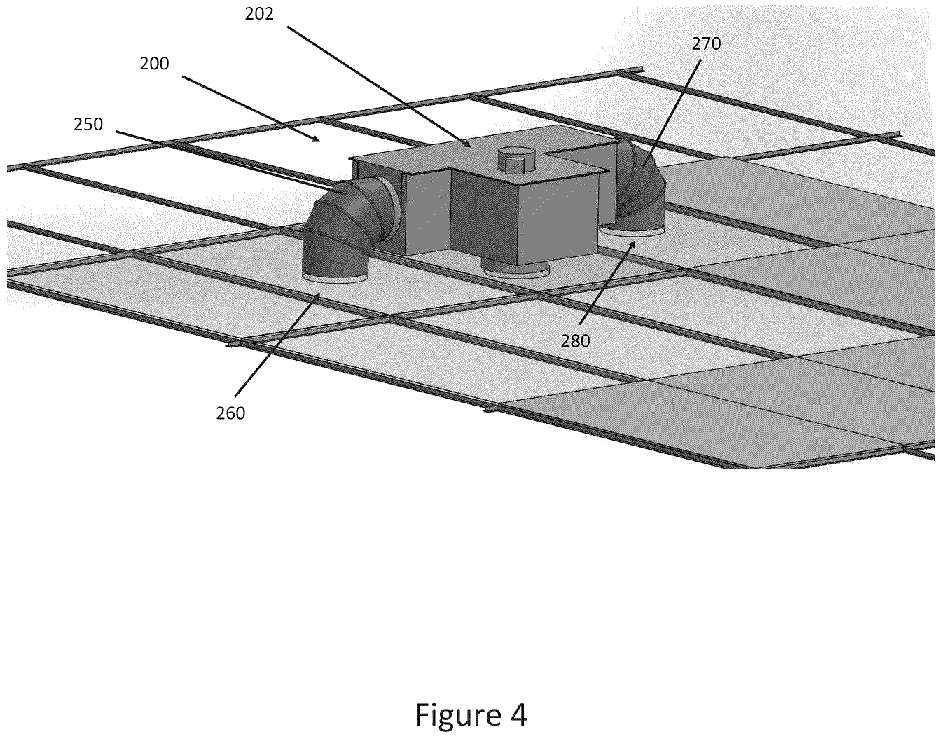

FIG. 3 shows an embodiment of an AAQC unit 200. The AAQC unit 200 may be mounted in a building and configured to filter the air. The AAQC unit 200 comprises a plurality of mitigation modules 9 which can be turned on and off as described above in relation to FIGS. 1 and 2. The AAQC unit 200, and the plurality of mitigation modules 9 can be controlled by the control computer 100, also referred to as a controller or control system, which can have processing electronics configured to control operation of the components of the unit 200. The plurality of mitigation modules can be turned on and off based on sensor input and other inputs by the control computer 100. The AAQC unit 200 can comprise an air input handler 210 and a manifold 231 defining at least a portion of an air path 230. The air input handler 210 may include an air mover such as a blower configure to draw air out of a room and force air into the air path 230. Once in the air path 230 the air is treated, and then exits the AAQC unit 200. The manifold 231 can include a first side tube 250 and a second side tube 270, however it should be understood that the manifold 231 need not include two sides. It should further be understood that although referred to as tubes 250,270, the tubes may be any type of manifold, including a variety of shapes such as square, rectangular, octagonal, or hexagonal. The first side tube 250 may comprise straight portion 252 and a bend 254. The second side tube 270 may comprise straight portion 272 and a bend 274. The air path can further include a splitter 234, wherein the splitter 234 is configured to split the air between the first side tube 250 and the second side tube 270. The downstream side of the splitter 234 is connected to the upstream side of the first tube 250 and/or the upstream side of the second tube 270. The splitter 234 may be located or connected to the junction between the first side tube 250 and the second side tube 270. The upstream side of the splitter 234 is connected to the air input handler 210. The first side tube 250 can comprise a first side outlet 260 located on a downstream side of the first side tube 250. The second side tube 270 can comprise a second side outlet 280 located on the downstream side of the second side tube 270. The first side outlet 260 and the second side outlet 280 can return air to the same room or environment from which the inlet 211 takes air. FIG. 4 shows an alternative embodiment of the AAQC unit 200 which further includes a housing 202. The housing 202 may contain all or a portion of the air input handler 210. The housing 202 may further contain all or a portion of the air path 230.

As shown in FIG. 5, there can be a plurality of internal components within the air path 230. An air path 230 may be defined by an air pathway extending from at least one inlet 211 to at least one outlet 260, 280. In the illustrated embodiment, the air path 230 extends from the inlet 211 through the first side tube 250 and the second side tube 270, and then out the first side outlet 260 and the second side outlet 280 back into the room or building. The manifold 231 can include an internal air guide 232, which comprises an auger 233. The auger 233 may also be referred to as a helical or spiral baffle. Although not visible in FIG. 5, the air path 230 further comprises a UV-C lamp 238 configured to kill viruses, bacteria, fungi, and other organic and inorganic contaminants. The internal air guide 232 may comprise a helical air guide (which also may be referred to as an auger) configured to extend the path the air takes from the air input handler 210 to an outlet such as the first side outlet 260 or the second side outlet 280. The internal air guide 232 may be a single part or may be constructed out of multiples parts or portions, for example a first part could be located on a first side tube 250 and a second part could be located on a second side tube 270. The internal air guide 232 and the inside surface of the first side tube 250 or the second side tube 270 together may define a spiral air path 237. The spiral air path 237 may define the treatment air path 236.

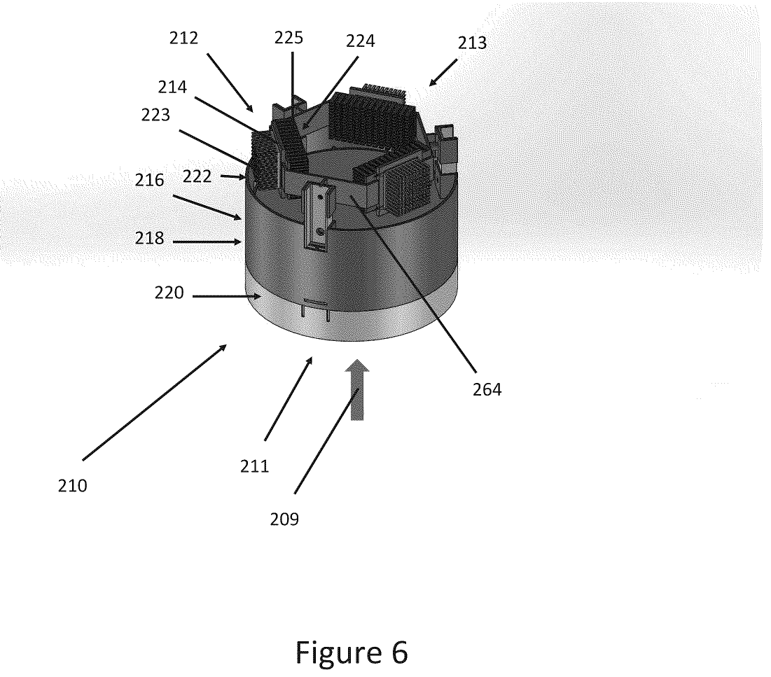

FIG. 6 shows an embodiment of a portion of air input handler 210 comprising an air inlet 211 and an air input handler outlet 213. The flow path of the air through the air input handler 210 enters via the inlet 211 and exits via the air input handler outlet 213. The arrow 209 represents the direction of flow through the air input handler 210. As the flows through the air input handler 210 it flows past a plurality of hydroxyl generators 212. The air input handler 210 can be used in conjunction with any of the embodiments disclosed herein. The air input handler 210 can comprise a hydroxyl generator 212, which is an example of a mitigation module 9. The hydroxyl generator 212 can be positioned upstream of the UV-C lamps 238 such that water vapor is entrained with the air when the air reaches the light emitted from UV-C lamps 238, another mitigation module. The generation of the OH radicals occurs during the interaction of the UV energy with water molecules entrained with the air that is driven along the treatment air path 236. The OH radicals contribute to the destruction of long chain molecules present in breathing air, most of which are too dangerous for human consumption. The present embodiment illustrates a plurality of (e.g., three) hydroxyl generator 212, but it should be understood that an AAQC unit 200 may comprise any number of hydroxyl generators 212.

The hydroxyl generator 212 is configured to scavenge water from the air. The hydroxyl generator 212 may collect water from the air during operation or during off hours. The hydroxyl generator 212 may comprise a Peltier cooler 214. A Peltier cooler, which may also be referred to as a thermoelectric heat pump or thermoelectric cooler, is a solid-state active heat pump which transfers heat from one side of the device to the other. Peltiers are powered devices leverage a temperature difference that is created by applying a voltage between two electrodes connected to a sample of semiconductor material. The devices may use n- and p-doped materials that are arranged to create a thermal gradient when powered on.

The hydroxyl generator 212 may further comprise a cold heat sink 222 and a hot heat sink 224 connected on either side of the Peltier cooler 214. The cold heat sink 222 can comprise a plurality of fins 223 and the hot heat sink 224 can comprise a plurality of fins 225. It should be understood that the fins 223, 225 may be poles, stakes, standoffs, or any other type of protrusion extending away from the base of the part. The Peltier cooler 214 of the hydroxyl generator 212 is configured to cool the cold heat sink 222 below the dew point, at which point water will collect on the fins 223 of the cold heat sink 222. The air moving across the cold heat sink 222 picks up the water that has collected on the fins 223 of the cold heat sink 222. The plurality of fins 223 are oriented perpendicular to the flow of air through the air input handler 210 to better transfer the accumulated water. In the present embodiment the three hydroxyl generators 212 are vertically oriented such that the fins are perpendicular to the flow. The plurality of hydroxyl generators 212 are spaced a distance apart from one another and are mounted on a hydroxyl generator stand 264. The hydroxyl generator stand holds the plurality of hydroxyl generator 212 such that there is a gap between the hydroxyl generator 212 and the filters 216,218. The hydroxyl generator stand 264 may form a close shape wherein the cold heat sink 222 is located on an exterior of the closed shape and the hot heat sink 224 is located on an inside of the closed shape. The Peltier cooler 214 may be integrated into the hydroxyl generator stand 264 such that each side of the Peltier cooler 214 is connected to a portion of the hydroxyl generator stand 264. In this case the Peltier cooler 214 is directly connected to the cold heat sink 222 and the hot heat sink 224. Alternatively, the Peltier cooler 214 may be mounted on either side of the hydroxyl generator stand 264, in which case the thermal transfer facilitated by the Peltier cooler 214 between the cold heat sink 222 and the hot heat sink 224 may be conducted through the hydroxyl generator stand 264. In that type of embodiment, the Peltier cooler 214 may be indirectly connected to the cold heat sink 222 or the hot heat sink 224. Although not shown, the hydroxyl generator 212 may further comprise a water collection holder. The hydroxyl generator 212 may be configured to collect water during operation of the AAQC unit 200, during times in which the AAQC unit 200 is running an alternative state such a water recuperation mode, or when the AAQC unit 200 is off. The water collected during these times may be stored in the water collection holder for future use to entrain water vapor with air flowing along pathway 236. As previously discussed, the water vapor can be energized by UV-C radiation, creating hydroxyls which can destroy various types of organic contaminants. The hydroxyl generator 212 is one of a plurality of mitigation modules and as such, may be controlled by the control computer 100. The control computer may rely on sensor 18 data, to determine whether the hydroxyl generator 212 should be on or off, and further determinations such as the ideal operation temperature of the cold heat sink 222 to best scavenge water based on the current temperature, humidity, etc.

The air input handler 210 may further comprise a HEPA filter 216 or a granular activated carbon (GAC) filter 218. In some embodiments the HEPA filter 216 and the GAC filter 218 may be one unit, as shown in FIG. 6. The air input handler 210 may further comprise a large contaminant air prefilter 220 configured to filter out larger particles. The air flows through the large contaminant air prefilter 220, then through the HEPA filter 216 and/or the GAC filter 218, and then past the hydroxyl generator 212.

As explained above, the internal air guide 232 may be helical in shape and configured to extend the path of the air flowing through AAQC unit 200. The internal air guide 232 and the walls of the first side tube 250 or the second side tube 270 can be lined with high UV-C reflectivity (98%) polytetrafluoroethylene (PTFE) and titanium dioxide (TiO₂) surface coatings. The coatings can cooperate with the UV-C lamp 238 to transform water molecules into hydroxyl OH radicals. The titanium dioxide LACO, high power UV-C radiation and long irradiation path length generate hydroxyl OH radicals that break the atomic bonds of organic and inorganic contaminants, delivering a single pass kill dose, inactivating and destroying a wide range of viruses, bacteria, and other pollutants in the process.

In some embodiments, input air may be filtered using a prefilter in combination with a high efficiency particulate air (HEPA) filter, just ahead of or just behind the blower. The system may be assembled into a generally round tubing or rectangular duct configurations.

FIG. 7 shows an embodiment of AAQC unit 200 comprising a plurality of internal components. The air path 230 can comprise a flow splitter 234 that splits the flow between the first side tube 250 and the second side tube 270. The air path 230 can further include the internal air guide 232 inside of the first side tube 250 or the second side tube 270. The air may flow from the underlying room or building through the inlet 211, through air input handler 210, and into the flow splitter 234. The flow splitter 234 may then split the air evenly between the first side tube 250 and the second side tube 270. The air may pass through the first side tube 250 or the second side tube 270. In the first side tube 250 or the second side tube 270 the air is forced to follow a longer path created by the internal air guide 232. The longer air path created by the internal air guide 232 within the first side tube 250 or the second side tube 270 may be referred to as the treatment air path 236. The internal air guide 232 creates a long treatment air path 236 which increases the dwell time of the air, e.g., the time for which the air is exposed to UV light from the UV lamps 238. An increased dwell time allows the AAQC unit 200 to more thoroughly and efficiently treat air passing through the AAQC unit 200. As such, the AAQC unit 200 is able to achieve a single pass kill in which 99% or more of viruses are killed in a single pass of air through the AAQC unit 200. In contrast, traditional systems require multiple cycles of air through the system in order to achieve the kill rate of the present disclosure. The dwell time of the AAQC unit is significantly longer than traditional systems due to, for example, the unit's 200 internal air guide which extends the air path, increasing dwell time, and as such increase the kill percentage.

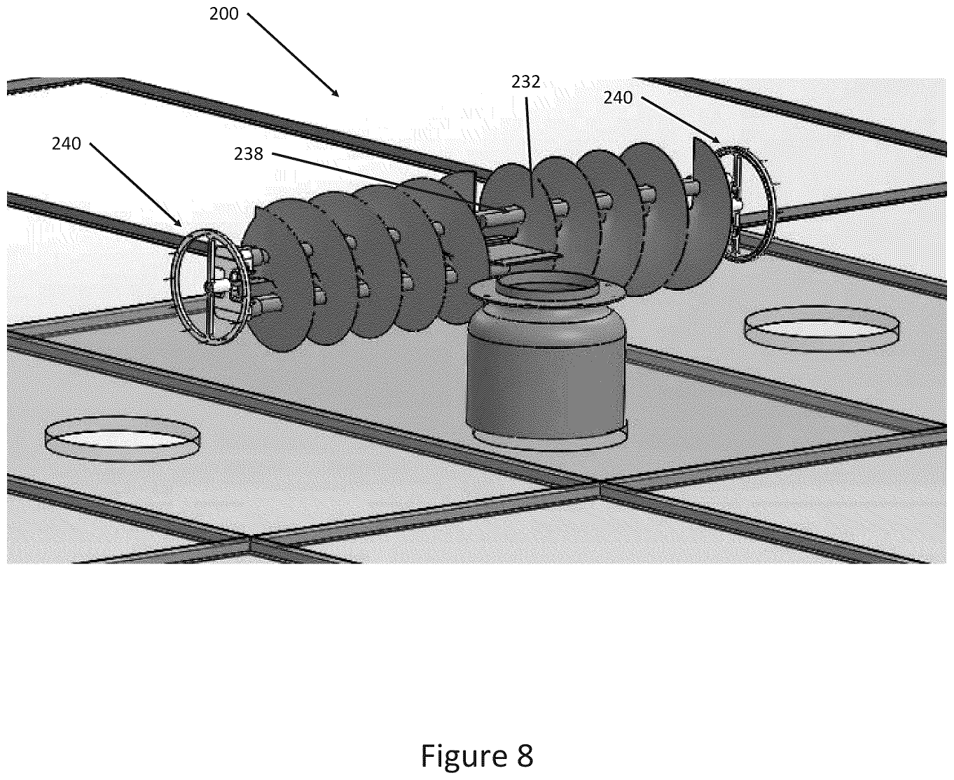

FIG. 8 shows an embodiment of a portion of the AAQC unit 200 shown in FIG. 7. The air path 230 can further comprise UV-C lamps 238 and an ION generator 240. An ION generator acts by charging the particles in a room so that they are attracted to walls, floors, tabletops, draperies, occupants, etc., resulting in fewer particles in the air. While the air travels through the treatment air path 236 the air is treated by the UV-C lamps 238. The internal air guide 232 forces the air into a spiral path 237, which increase the dwell time or exposure time of the air to the UV-lamps. There may be a single UV-C lamp 238 or multiple UV-C lamps 238 as shown in the present embodiment. The lamps 238 may run parallel to the straight portion 252 of the first side tube 250 and the straight portion 272 of the second side tube 270. In the present embodiment the UV-C lamps 238 extend longitudinally in an orientation generally parallel to the flow pathway. The UV-C lamps 238 may extend the full length of the first side tube 250 or the second side tube 270 or may extend a portion of the length. In the present embodiment the UV-C lamps 238 extend the full length of a straight portion 252 of the first side tube 250 and a full length of the straight portion 272 of the second side tube 270 as shown in FIG. 7. The multiple UV-C lamps 238 may be located in the center of the first side tube 250 or the second side tube 270. The UV-C lamps 238 may be located in relation to the tubes 250,270 or to the internal air guide 232, or the auger 233. The UV-C lamps 238 may be parallel to the axis of the auger 233. The UV-C lamps 238 may be located on or near the axis of the 233. The UV-C lamps 238 may also be parallel to the axis of the auger 233 but spaced a distance from the axis of the auger 233. Each of the UV-C lamps 238 may be spaced out circumferentially at a set distance from the auger 233. In the present embodiment the UV-C lamps 238 are grouped into two pairs, wherein each pair is parallel to the axis of the auger 233 and spaced a distance away from the axis of the auger 233. In the present embodiment the UV-C lamps 238 pairs are located 180 degrees apart from each other for a set distance in relation to the axis of the auger 233. The UV-C lamps 238 may be mounted directly to the auger 233 or may only pass through the auger 233.

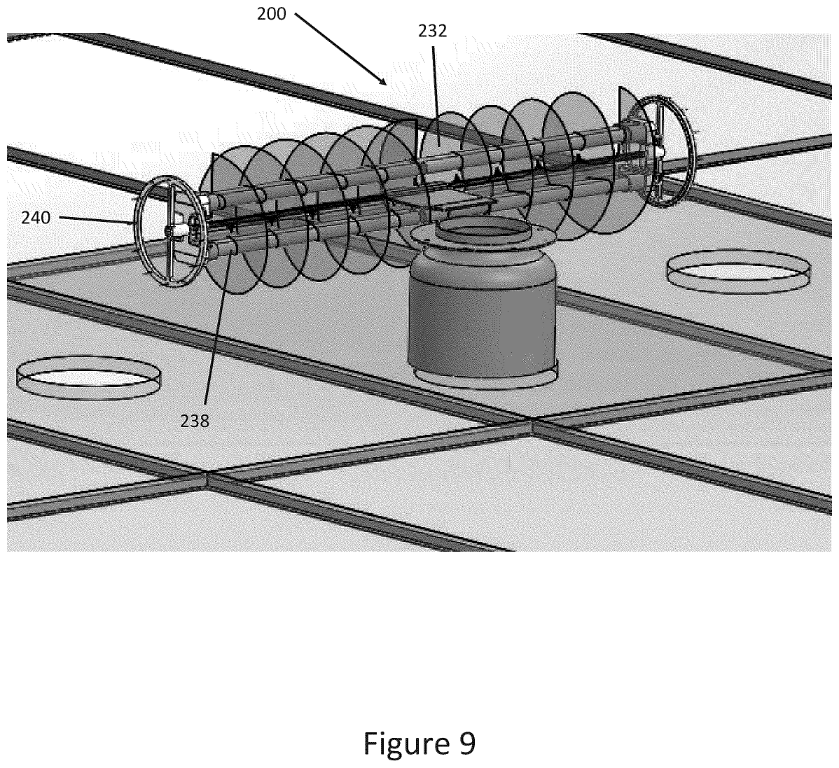

The air within the air path 230 may further be treated by the ION generator 240. There may be an ION generator 240 located in both the first side tube 250 and the second side tube 270. The ION generator 240 can be located within the AAQC unit 200 such that the air has already been substantially treated. The ION generator 240 can be located within the air path 230 at a location where the air has already been treated by the UV-C lamps 238. For example, the illustrated ION generator 240 can be downstream of the UV-C lamps 238. FIG. 9 shows the AAQC unit of FIG. 8 with the internal air guide 232 translucent, so as the better see the UV-C lamps 238 and the ION generator 240.

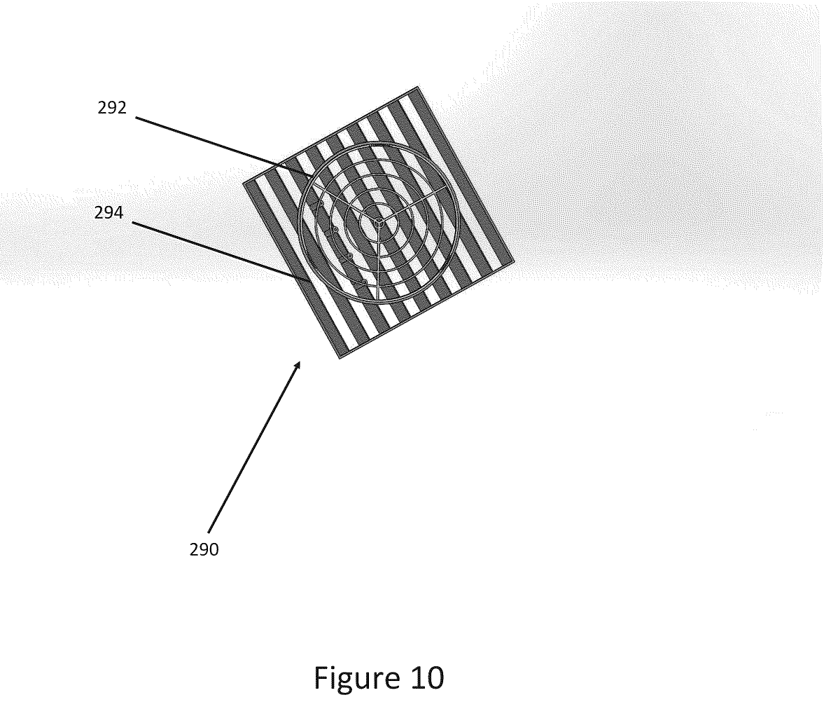

FIG. 10 shows an embodiment of an output air diffuser 290. The AAQC unit 200 can comprise an output air diffuser 290 at both the first side outlet 260 and the second side outlet 280. The output air diffuser 290 may be configured to control the flow shape and direction of the air being discharged from the air path 230. The horizontal slats 294 may be angled so as to direct air in a certain direction. The ION generator 240 may further comprise ION brushes 292. The ION brushes 292 may comprise a rounded metal structure, having multiple concentric circular segments connected by radially-extending spokes. The ION brushes 292 may further be carbon fiber brushes that are high electric field points, enabling greater flux of ions. The ION brushes 292 may be directly connected to the output air diffuser 290 but need not be. The ION brushes 292 may be connected to the ION generator and configured to charge particles. Locating the ION brushes 292 as downstream as possible reduces the ability of particle to cling to the inside structure of the AAQC unit. It should further be understood that the ION generator 240 may also be mounted in direct proximity to the output air diffuser 290 in alternative embodiments.

FIG. 11 shows an alternative embodiment of a AAQC unit 300. The AAQC unit 300 comprises a plurality of mitigation modules 9 which can be turned on an off as described above in relation to FIGS. 1 and 2. The plurality of mitigation modules 9 can be turned on and off based on sensor input and other inputs. The AAQC unit 300 may be mounted inside of a building, a bus, a train, an airplane, or any suitable location in which it is desirable to remove contaminants from the air. The unit may be mounted onto a surface in a manner similar to that of a light or bathroom vent fan.

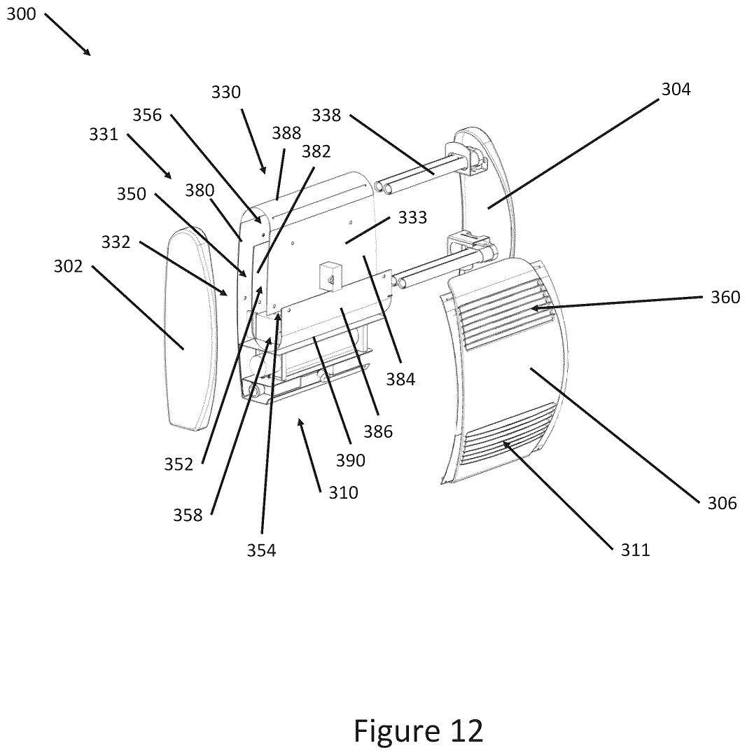

FIG. 12 shows an exploded view of the AAQC unit 300 of FIG. 11. The AAQC unit 300 can comprise a first side panel 302, a second side panel 304, and a front cover 306. The AAQC unit 300 includes internal air guide 332, which may be comprise an baffle 333, at least partially defining an air path 330. The AAQC 300 comprises a manifold 331 that can define at least a portion of the air path 330 which extends from the inlet 311 to the outlet 360. The baffle 333 can further include or define a portion or all of the internal air guide 332. The AAQC unit 300 further comprise an ultraviolet (e.g., UV-C) lamp 338 which may be located within the air path 330.

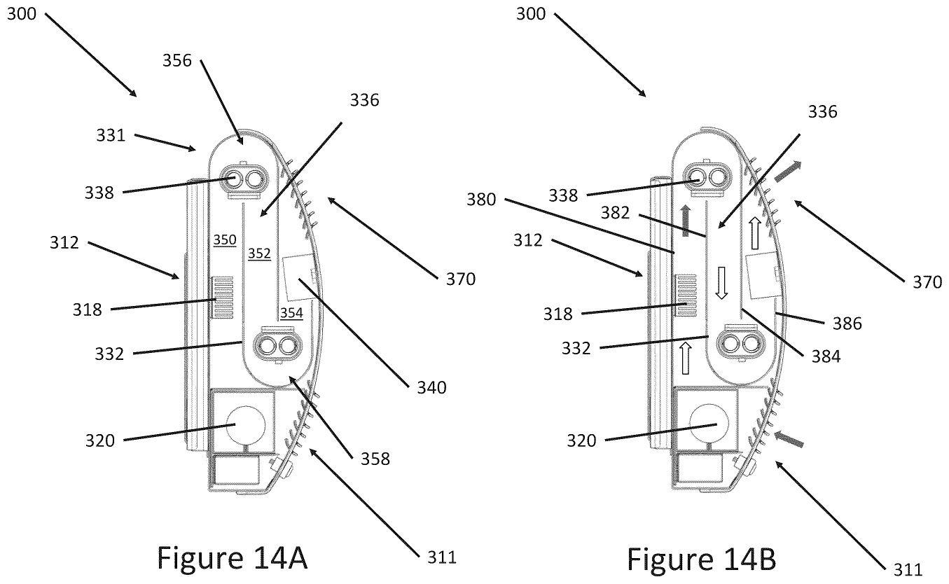

The internal air guide 332 may create a serpentine air path configured to extend the path of the air flowing through AAQC unit 300. The internal air guide 332 may at least partially define the treatment air path 336. The internal air guide 332 may comprise a first wall 380, a second wall 382, a third wall 384, and a fourth wall 386. All of the walls may be substantially parallel to one another however that need not be the case. The plurality of walls may not be parallel to one another and may create passage ways varying in size and orientation. The first wall 380 may be connected to the third wall 384 via a first bend wall 388. The first bend wall 388 can comprise a curved surface that redirects the air back 180 degrees, however it should be understood that the bend may not be a curve and may be any angle, so long as it redirects the air. The second wall 382 may be connected to the fourth wall 386 via the second bend wall 390. The second bend wall 390 can comprise a curved surface that redirects the air back 180 degrees, however it should be understood that the bend may not be a curve and may be any angle, so long as it redirects the air. As such, the internal air guide 332 may comprise two individual parts, one comprising the first wall 380, the first bend wall 388, and the third wall 384. The other comprising the second wall 382, the second bend wall 390, and the fourth wall 386. The treatment air path 336 may comprise a first passage 350 defined by a first wall 380 and a second wall 382, a second passage 352 defined by a second wall 382 and a third wall 384, and a third passage 354 defined by a third wall 384 and a fourth wall 386. The first passage 350 may be connected to the second passage 352 by a first bend passage 356. The first bend passage 356 may be defined by the first bend wall 388. The second passage 352 may be connected to the third passage 354 by the second bend passage 358. The plurality of walls and passages may also be seen in FIGS. 14A-14B. The air in the treatment air path may flow from the first passage 350, through the first bend passage 356, through the second passage 352, through the second bend passage 358, and then through the third passage 354.

The internal air guide 332 and the internal surfaces of the AAQC unit 300 can be lined with high UV-C reflectivity (98%) polytetrafluoroethylene (PTFE) and titanium dioxide (TiO₂) surface coatings. The coatings can absorb the energy of the UV-C lamps 338 to work with the UV-C lamps 338 to convert scavenged water molecules into hydroxyl OH radicals. The hydroxyl OH radicals can destroy or kill various viruses, bacteria, fungi, and other airborne risks.

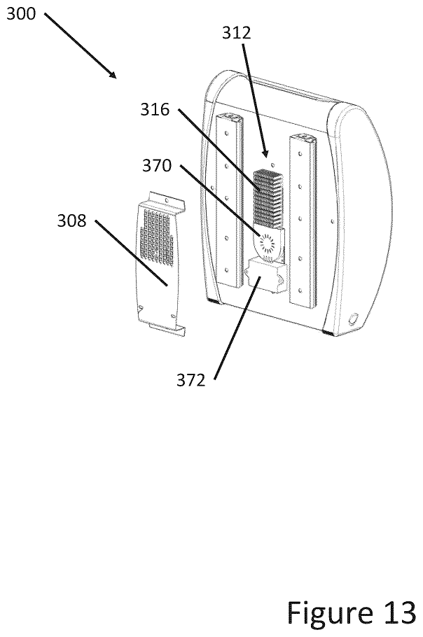

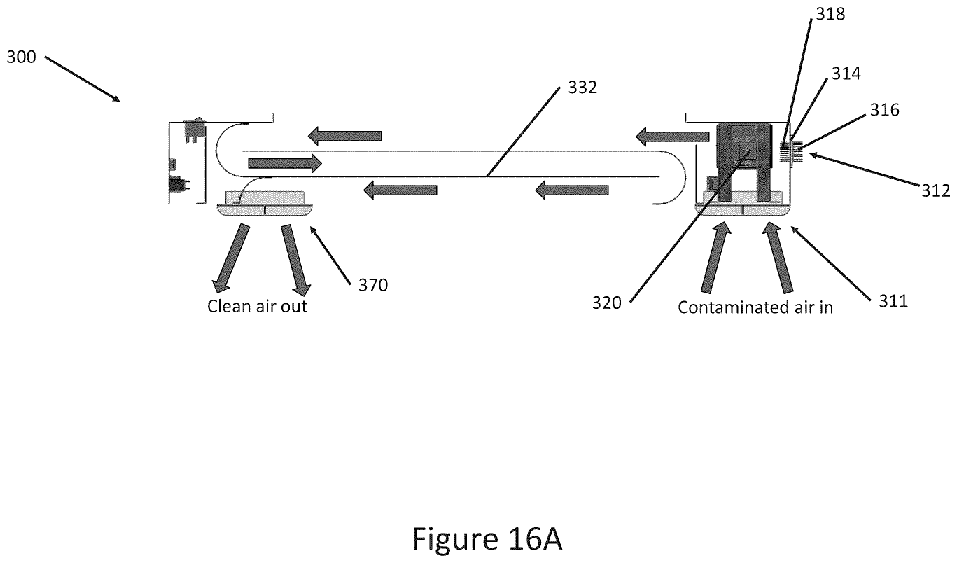

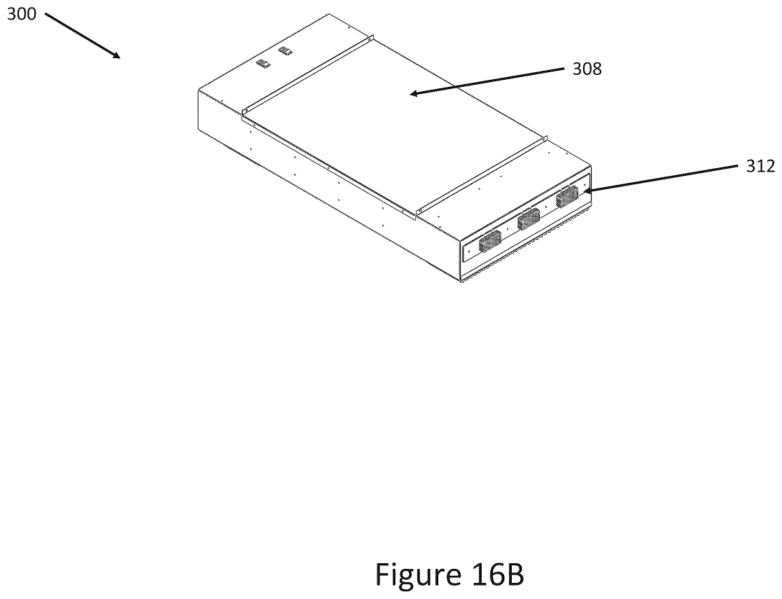

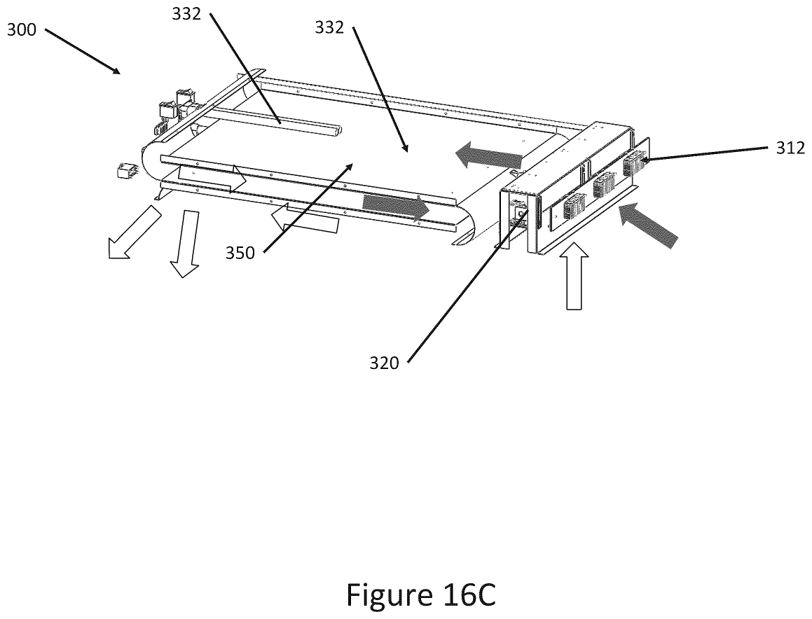

FIG. 13 shows a view of the back of the AAQC unit 300. The AAQC unit 300 can further comprise an access cover 308, a cooling blower 370, and a power converter 372, which may also be referred to as a power supply, any of which may be mounted on the back of the AAQC unit 300. The AAQC unit 300 may further comprise a hydroxyl generator 312. The hydroxyl generator 312 can comprise a hot heat sink 316, a cold heat sink 318, and a thermoelectric cooler 314 (e.g., a Peltier cooler) as shown in FIG. 16A. All of the hydroxyl generator 312 or a portion thereof, for example, the cold heat sink 318 may be located within the air path 330. In preferred embodiments the cold heat sink 318 is located within the air path 330 and the hot heat sink 316 is not located within the air path 330. The cooling blower 370 may blow on the hot heat sink 316 to better dissipate heat generated on the hot heat sink 316 as the Peltier cooler 314 cools the cold heat sink 318.

FIGS. 14A and 14B show a cutaway side view of the AAQC unit 300. The contaminated air enters at the inlet 311. Although not shown there may be a plurality of filters such as a HEPA filter, a GAC filter, or a large contaminant air prefilter directly upstream or downstream of the inlet 311. The filters can be located near, next to, or proximate to the inlet 311. The AAQC unit 300 may further include a cross flow fan 320. The air may flow from the inlet 311, through the cross-flow fan 320, and then past the cold heat sink 318. As the air passes the cold heat sink 318 it can pick up water vapor so long as the cold heat sink 318 is at or below the dew point. As previously described, generation of the OH radicals occurs during the interaction of the UV energy with water molecules present in the ambient atmosphere. These radicals contribute to the destruction of long chain molecules present in breathing air, most of which are too dangerous for human consumption. After the air passes the cold heat sink 318 the air passes into enters the treatment air path 336 defined by internal air guide 332.

The treatment air path 336 may be fully or partially defined by the internal air guide 332. The UV-C lamps 338 may be located within the treatment air path 336. For example, the UV-C lamps 338 may be located within the first bend passage 356 and/or the second bend passage 358. It should be understood that the UV-C lamps 338 may be partially located within the first passage 350, second passage 352, or third passage 354. The UV-C lamps 338 can be perpendicular (90 degrees) to the first passage 350, second passage 352, or third passage 354 as shown in the illustrated embodiment. The UV-C lamps 338 may also have alternative orientations to the plurality of passages 350, 352, 354. For example, the UV-C lamps 338 may be oriented at an angle slightly different than perpendicular, for example an angle of 85 degrees or 95 degrees to the plurality of passages 350, 352, 354. The UV-C lamps 338 may further be oriented at a variety of angles, for example an angle of 30 degrees or 45 degrees to the plurality of passages 350, 352, 354. The air may exit the treatment air path 336 and pass by an ION generator 340 which charges any particles which then pass through the outlet 360. FIG. 14B shows the same components of 14A, in addition to a plurality of arrows indicating the flow of the air.

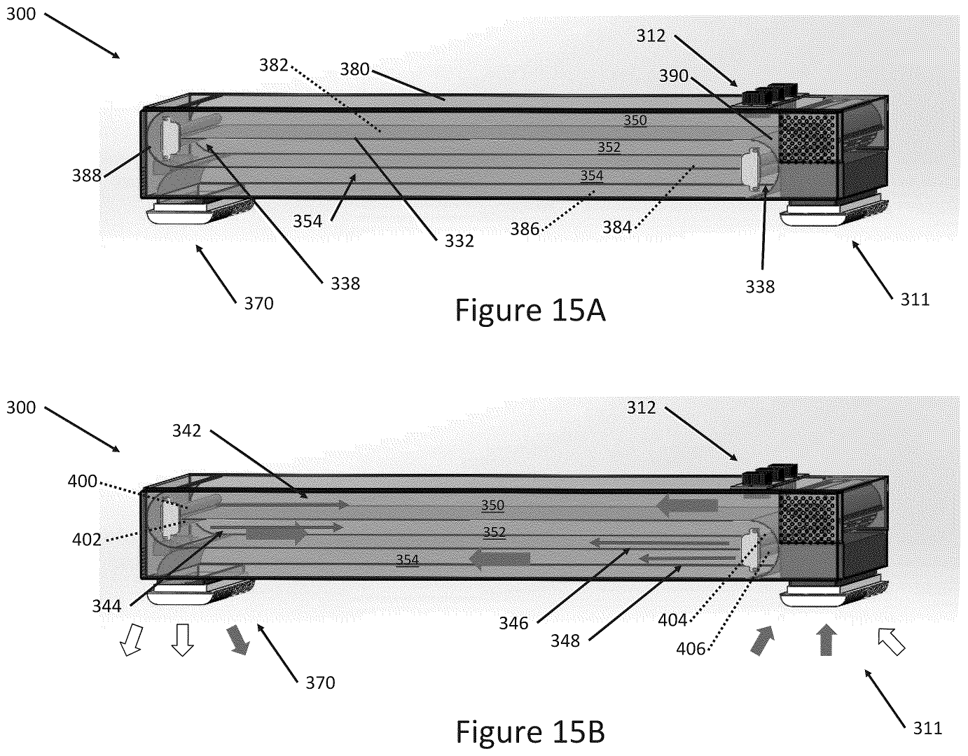

FIG. 15A show an alternative embodiment of the AAQC unit 300. The AAQC unit 300 comprises three generators 312. The hydroxyl generators 312 are located upstream of the treatment air path 336 and the UV-C lamps 338 so that there is water vapor in the air prior to the airs exposure to the light generated by the UV-C lamps 338. The embodiment of the AAQC unit 300 further comprises a plurality of (e.g., four) UV-C lamps 338. The plurality of UV-C lamps may comprise a first UV-C lamp 400, a second UV-C lamp 402, a third UV-C lamp 404, and a fourth UV-C lamp 406. The UV-C lamps 338 expose air within the treatment air path 336 to UV light which destroys and/or kills contaminants. The arrow 342 shown in FIG. 15B indicate the UV-C radiation path within the treatment air path 336 generated by a first UV-C lamp 400 of the plurality of UV-C lamps 338. The light emitted by the first UV-C lamp 400 can propagate down the first passage 350. The light emitted from the second UV-C lamp 402 and the third UV-C lamp 404 can propagate along the second passage 352. The arrow 344 indicates the UV-C radiation path of the second UV-C lamp 402 and the arrow 346 indicates the UV-C radiation path of the third UV-C lamp 404. The light emitted from the fourth UV-C lamp 406 irradiates the third passage 354. The arrow 348 indicates the UV-C radiation path of the third UV-C lamp 404. Each of the plurality of UV-C lamps 338 are located in the first bend passage 356 and the second bend passage 358, however as seen in FIGS. 15A and 15B they are substantially in line with their respective passageways (350, 352, 354). The light emitted from the UV-C lamp 338 is reflected off the internal air guide 332 as well as other components located within treatment air path 336. As such, the arrows 342 may generally define a light path but it should be appreciated that the light emitted from the UV-C lamps 338 will reflect and/or scatter off the internal walls along the pathway 336.

FIG. 16A-C shows an alternative embodiment of the AAQC unit 300 comprising three hydroxyl generators 312 located on a side of the AAQC unit 300. The hydroxyl generator 312 comprises the Peltier cooler 314, the hot heat sink 316, and the cold heat sink 318. The hot heat sink 316 is connected to a first side of the Peltier cooler 314 and the cold heat sink 318 is connected to a second side of the Peltier cooler 314. The cold heat sink 318 is located on the interior of the AAQC unit 300 and the hot heat sink 316 is located on the exterior of the AAQC unit 300. This configuration allows for the maximum water scavenging by the hydroxyl generator 312. The air flows in through the inlet 311, by the hydroxyl generator 312, and then into the crossflow fan 320. FIG. 16C shows an alternative orientation and location for the UV-C lamps 338. The UV-C lamps 338 are located parallel to and within the first passage 350.

Although this disclosure has been described in the context of certain embodiments and examples, it will be understood by those skilled in the art that the disclosure extends beyond the specifically disclosed embodiments to other alternative embodiments and/or uses and obvious modifications and equivalents thereof. In addition, while several variations of the embodiments of the disclosure have been shown and described in detail, other modifications, which are within the scope of this disclosure, will be readily apparent to those of skill in the art. It is also contemplated that various combinations or sub-combinations of the specific features and aspects of the embodiments may be made and still fall within the scope of the disclosure. For example, features described above in connection with one embodiment can be used with a different embodiment described herein and the combination still fall within the scope of the disclosure. It should be understood that various features and aspects of the disclosed embodiments can be combined with, or substituted for, one another in order to form varying modes of the embodiments of the disclosure. Thus, it is intended that the scope of the disclosure herein should not be limited by the particular embodiments described above. Accordingly, unless otherwise stated, or unless clearly incompatible, each embodiment of this invention may comprise, additional to its essential features described herein, one or more features as described herein from each other embodiment of the invention disclosed herein.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a sub combination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount.

The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted fairly based on the language employed in the claims and not limited to the examples described in the present specification or during the prosecution of the application, which examples are to be construed as non-exclusive.

What is claimed is:

1. An adaptive air quality control system comprising:
a manifold comprising an air guide, the air guide directing air along an air pathway during operation of the adaptive air quality control system;
a hydroxyl generator positioned along the air pathway and configured to scavenge water from the air; and
an ultraviolet (UV) lamp positioned along the air pathway, the UV lamp configured to irradiate the air along the air pathway during operation of the adaptive air quality control system.

2. The adaptive air quality control system of claim 1, further comprising a blower configured to drive air flow through the system.

3. The adaptive air quality control system of claim 1, further comprising a filter, wherein with the filter is upstream of the hydroxyl generator.

4. The adaptive air quality control system of claim 1, wherein the hydroxyl generator is upstream of the UV lamp.

5. The adaptive air quality control system of claim 1, further comprising an ION generator, wherein the ION generator is downstream of the UV lamp.

6. The adaptive air quality control system of claim 1, wherein the air guide comprises one or more curved or angled portions defining a serpentine air path.

7. The adaptive air quality control system of claim 1, wherein the air guide comprises one or more curved or angled portions defining a helical air path.

8. The adaptive air quality control system of claim 7, wherein the helical air path forms a spiral and the UV lamp is located within the spiral.

9. The adaptive air quality control system of claim 1, wherein air guide comprises a coating comprising polytetrafluoroethylene (PTFE) or titanium dioxide (TiO2).

10. The adaptive air quality control system of claim 1, wherein the hydroxyl generator comprises a thermoelectric cooler, a power supply, and a heat exchanger.

11. The adaptive air quality control system of claim 1, wherein the hydroxyl generator is configured to be chilled below a dew point to accumulate moisture.

12. An adaptive air quality control system comprising:
at least one ultraviolet (UV) lamp;
an internal air guide comprising:
a first passage;
a second passage parallel to the first passage; and
a bend passage connecting the first passage to the second passage and configured to redirect air flowing through the first passage into the second passage;
wherein the UV lamp is within or exposed to the internal air guide to emit light within one or both of the first passage and second passage.

13. The adaptive air quality control system of claim 12, wherein the UV lamp is located adjacent to the bend passage and configure to emit a light within the first passage or the second passage.

14. The adaptive air quality control system of claim 12, wherein the internal air guide further comprises a first wall, a second wall, and a third wall;
wherein the first passage is defined by the first wall and the second wall; and
wherein the second passage is defined by the second wall and the third wall.

15. The adaptive air quality control system of claim 14, wherein the first wall or the second wall comprises a coating.

16. The adaptive air quality control system of claim 12, further comprising a hydroxyl generator located upstream of the UV lamp.

17. An adaptive air quality control system comprising:
a manifold;
at least one ultraviolet (UV) lamp located within the manifold;
a helical internal air guide located within the at least manifold;
wherein the helical internal air guide at least partially defines a spiral air pathway around the at least one UV lamp.

18. The adaptive air quality control system of claim 17, wherein the helical internal air guide comprises a coating.

19. The adaptive air quality control system of claim 18, wherein the coating comprises polytetrafluoroethylene (PTFE) or titanium dioxide (TiO2).

20. The adaptive air quality control system of claim 17, wherein the at least one UV lamp is parallel to an axis of the helical internal air guide.
